(19) United States
(12) Patent Application Publication
(10) Pub. No.: US 2014/0366905 A1
(43) Pub. Date: Dec. 18, 2014

(54) PROCESS FOR TREATING KERATIN FIBERS

(71) Applicants: Maxime De Boni, Mumbai (IN); Francoise Pataut, Paris (FR)

(72) Inventors: Maxime De Boni, Mumbai (IN); Francoise Pataut, Paris (FR)

(73) Assignee: L'OREAL, Paris (FR)

(21) Appl. No.: 14/374,332

(22) PCT Filed: Jan. 26, 2012
(86) PCT No.: PCT/JP2012/052325

§ 371 Date: Jul. 24, 2014

(51) Int. Cl. A45D 7/04; A61Q 5/04; A61K 8/41; A61K 8/22; A61K 8/46; A61K 8/19

(52) U.S. Cl. CPC A45D 7/04; A61K 8/46; A61K 8/19; A61K 8/41; A61K 8/22; A61Q 5/04
USPC 132/204

(57) ABSTRACT

The present invention relates to a process for treating keratin fibers, preferably hair, comprising the steps of: (i) applying to the keratin fibers a reducing composition comprising at least one disulfide and at least one thiol, wherein the weight ratio of disulfide/thiol is from 0.35 to 0.75, inclusive, preferably 0.4 to 0.7, and more preferably 0.45 to 0.65; (ii) optionally rinsing the keratin fibers; (iii) oxidizing the keratin fibers; and (iv) optionally rinsing and/or drying the keratin fibers. The present invention provides a new treatment process, in particular a permanent deformation process, for keratin fibers such as hair, which can provide superior cosmetic effects such as long-lasting deformation, sufficient curl efficiency, reduced hair degradation and non-malodor, in a relatively short period of time.

### TECHNICAL FIELD

The present invention relates to a process for treating keratin fibers such as hair.

### BACKGROUND ART

Many hair care products are marketed nowadays to easily style, texturize and add some weight to the hair, especially to thinning hair, amongst which foams and styling gels or hair lacquers may be mentioned as examples. These products enable shaping of the hair but are removed with shampoo and thus are required to be applied on a daily basis.

The most general technique for obtaining long-lasting deformation of the hair consists, in a first step, of opening the keratin S—S disulfide bonds (cystine) with a composition comprising a suitable reducing agent (reducing step) then, once the thus treated hair has been rinsed, generally with water, reforming said disulfide bonds in a second step, by applying an oxidizing composition onto the hair which has been placed beforehand under tension, using curlers for example (an oxidizing step, also called a fixing step), so as to give the hair the desired form in the end. For example, WO 97/30683 discloses a standard process for permanent waving or straightening keratin fibers such as hair in line with the above steps.

The new shape that is imposed to the hair by means of a chemical treatment, such as explained above, is relatively long-lasting and is particularly resistant to washing operations with water or shampoo, as compared to the usual simple methods for temporarily reshaping the hair by using foams, styling gels, or lacquers.

Many compositions and processes for the above chemical treatment have been proposed. Generally, they offer good performances on the day of treatment.

### DISCLOSURE OF INVENTION

However, there are various drawbacks as follows in the above chemical treatment process that may not be suitable from the view-point of consumer's or hair-dresser's expectations:
Insufficient long-lastingness against environmental stress (mechanical constraints from brushing, frequent shampoos, light exposure, high humidity and the like);
Insufficient perm efficiency on natural hair;
High levels of hair degradation, especially in repeated applications or in combination with other chemical treatments such as oxidative coloration;
Long processing time; and
Malodor of thiol-compounds during and after the perm process.

In particular, the long processing time is important. The deformation process, in particular permanent waving or straightening, of keratin fibers such as hair requires a lot of time and constant attention by the hair-dresser. Reduction of the processing time is one of the keys in the development of deformation processes, such as permanent waving or straightening, of the keratin fibers. There is indeed a need to improve the deformation process of the keratin fibers to shorten the processing time without lowering the cosmetic performance of the deformation process.

Thus, an objective of the present invention is to provide a new treatment process, in particular a permanent deformation process, for keratin fibers such as hair, which can provide superior cosmetic effects such as long-lasting deformation, sufficient curl efficiency, reduced hair degradation and non-malodor, in a relatively short period of time.

The above objective of the present invention can be achieved by a process for treating keratin fibers, in particular hair, comprising the steps of:
(i) applying to the keratin fibers a reducing composition comprising at least one disulfide and at least one thiol, wherein the weight ratio of disulfide/thiol is from 0.35 to 0.75, inclusive, preferably 0.4 to 0.7, and more preferably 0.45 to 0.65;
(ii) optionally rinsing the keratin fibers;
(iii) oxidizing the keratin fibers; and
(iv) optionally rinsing and/or drying the keratin fibers.

The disulfide may be selected from the group consisting of dithiodiglycolic acid, dithiodilactic acid, cystine, cystamine, disulfide of mercaptopropionic acid, and salts thereof.

The disulfide may be present in the reducing composition in an amount of from 1 to 15% by weight, preferably 1.5 to 9% by weight, relative to the total weight of the composition.

The thiol may be selected from the group consisting of thioglycolic acid, thiolactic acid, cysteine, cysteamine, mercaptopropionic acid, and salts thereof.

The thiol may be present in the reducing composition in an amount of from 1 to 20% by weight, preferably 5 to 15% by weight, relative to the total weight of the composition.

It is preferable that the disulfide/thiol combination be chosen from the following combinations: dithiodiglycolic acid or its salts/thioglycolic acid or its salts, dithiodilactic acid or its salts/thiolactic acid or its salts, cystine/cysteine, cystamine/cysteamine, mercaptopropionic acid disulphide/mercaptopropionic acid.

The reducing composition may have a pH of from 7 to 10, preferably from 8 to 9.5.

It is preferable that the reducing composition comprise at least one alkali agent.

The alkali agent may be an inorganic alkaline agent.

The inorganic alkaline agent may be selected from the group consisting of ammonia, alkaline metal hydroxides, alkaline earth metal hydroxides, alkaline metal phosphates and alkaline metal monohydrogenophosphates, alkaline metal carbonates and alkaline metal monohydrogenocarbonates.

The alkali agent may be an organic alkaline agent.

The organic alkaline agent may be selected from the group consisting of monoamines and derivatives thereof, diamines and derivatives thereof, polyamines and derivatives thereof, basic amino acids and derivatives thereof, oligomers of basic amino acids and derivatives thereof, polymers of basic amino acids and derivatives thereof, urea and derivatives thereof; and guanidine and derivatives thereof.

The oxidation may be mild oxidation consisting of applying an oxidizing composition with a short, or with no, standing time on the keratin fibers.

It is preferable that the oxidizing composition comprise an aqueous hydrogen peroxide solution.

The concentration of the hydrogen peroxide in the oxidizing composition may range from 0.1 to 20% by weight, preferably 1 to 10% by weight, relative to the total weight of the oxidizing composition.

The oxidizing composition may be in the form of a shampoo, a gel, a mousse, a thickened lotion or a cream.

Thus, the present invention can provide a new treatment process, in particular a permanent deformation process, for keratin fibers such as hair, which can provide superior cosmetic effects such as long-lasting deformation, sufficient curl efficiency, reduced hair degradation and non-malodor, in a relatively short period of time.

### BEST MODE FOR CARRYING OUT THE INVENTION

After diligent research, the inventors have discovered that it is possible to reduce the processing time of a deformation process, in particular permanent waving or straightening, of keratin fibers such as hair, without deteriorating the cosmetic effects, such as long-lasting deformation, sufficient curl efficiency, reduced hair degradation and non-malodor, of the deformation process, by controlling the reducing power of a reducing agent to be used during the reducing step in the deformation process, thus allowing a much shorter time for applying an oxidizing composition during the fixing step.

Thus, the process for treating keratin fibers, preferably hair, according to the present invention comprises the steps of:
(i) applying to the keratin fibers a reducing composition comprising at least one disulfide and at least one thiol, wherein the weight ratio of disulfide/thiol is from 0.35 to 0.75, inclusive, preferably 0.4 to 0.7, and more preferably 0.45 to 0.65;
(ii) optionally rinsing the keratin fibers;
(iii) oxidizing the keratin fibers; and
(iv) optionally rinsing and/or drying the keratin fibers.

According to the present invention, the processing time required for treating the keratin fibers, in particular the oxidizing time of the keratin fibers, can be drastically shortened so that the performance of the deformation process of the keratin fibers can be facilitated with superior cosmetic effects such as long-lasting deformation, sufficient curl efficiency, reduced hair degradation and non-malodor.

The present invention will be described hereafter in a more detailed manner. In the following description the expression “at least one” is equivalent to the expression “one or several”.

(Reducing Composition)

The present invention uses a reducing composition comprising at least one disulfide. The expression “disulfide” here means a compound having at least one disulfide bond (—S—S—). As long as the disulfide includes at least one disulfide bond, the type of the disulfide is not limited. It is preferable to use a linear organic disulfide which has a linear chemical structure. The linear organic disulfide may be represented by the formula:

R¹—S—S—R²

wherein

R¹ and R² independently represent a linear, saturated or unsaturated hydrocarbon group, which may be interrupted by one or more hetero atoms selected from the group consisting of a sulfur atom, oxygen atom, nitrogen atom, silicon atom and phosphorous atom, and which may comprise one or more substituents selected from the group consisting of hydroxyl groups, cyano groups, ester groups, amino groups, amide groups, carbamoyl groups, carbamate groups, carboxylic acid groups, carbonate groups, hydrazinyl groups, ether groups, and ureido groups, and salts thereof.

It is possible that the linear organic disulfide compound be selected from the group consisting of dialkyldisulfides, dialkenyldisulfides, dialkylaryldisulfides, N,N′-dithiodialkylcarboxylic acids, N,N′-dithiodialkyl esters, N,N′-dithiodialkyl amines and N,N′-dithiodialkyl amides.

As the dialkyldisulfides, dialkenyldisulfides or diaryldisuffides, mention may be made of those represented by the following formula:

R³—(CH₂)ₓ—S—S—(CH₂)_{y}—R⁴

wherein

R³ and R⁴ independently denote a C₁-C₁₈ alkyl group, preferably a C₁-C₁₀ alkyl group, and more preferably a C₁-C₆ alkyl group; a C₂-C₁₈ alkenyl group, preferably a C₂-C₁₀ alkenyl group, and more preferably a C₂-C₆ alkenyl group; or a C₆-C₁₈ aryl group, preferably a C₆-C₁₂ aryl group, and more preferably a C₆-C₈ aryl group; and

x and y independently denote a number from 0 to 5, and preferably 0 to 3.

For example, dimethyl disulfide, diallyl disulfide, or diphenyl disulfide may be mentioned.

As the dicarboxydisulfides or dialkoxycarbonyldisulfides, mention may be made of those represented by the following formula:

R⁵OOC—(R⁶)ₛ—(CH₂)ₓ—S—S—(CH₂)_{y}—(R⁷)ₜ—COOR⁸

wherein

R⁵ and R⁸ independently denote a hydrogen atom; a C₁-C₁₈ alkyl group, preferably a C₁-C₁₀ alkyl group, and more preferably a C₁-C₆ alkyl group; a C₂-C₁₈ alkenyl group, preferably a C₂-C₁₀ alkenyl group, and more preferably a C₂-C₆ alkenyl group; or a C₆-C₁₈ aryl group, preferably a C₆-C₁₂ aryl group, and more preferably a C₆-C₈ aryl group;

R⁶ and R⁷ independently denote a C₁-C₁₈ alkylene group, preferably a C₁-C₁₀ alkylene group, and more preferably a C₁-C₆ alkylene group; a C₂-C₁₈ alkenylene group, preferably a C₂-C₁₀ alkenylene group, and more preferably a C₂-C₆ alkenylene group; or a C₆-C₁₈ arylene group, preferably a C₆-C₁₂ arylene group, and more preferably a C₆-C₈ arylene group, which may be substituted with one or more halogens and/or hydroxyl groups;

s and t independently denote a number from 0 to 2, and preferably 0 or 1;

x and y independently denote a number from 0 to 5, and preferably 0 to 3, and

each of s+x and y+t is higher or equal to 1, and

salts thereof.

For example, 3,3′-dithiodipropionic acid (disulfide of mercaptopropionic acid) represented by the following formula:

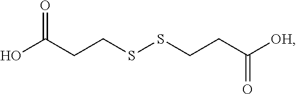 [SMILES: O=C(O)CCSSCCC(=O)O]

2,2′-dithiodipropionic acid (dithiodilactic acid) represented by the following formula:

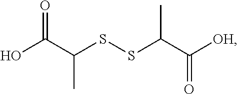

dithiodiglycolic acid, dithiodisalicylic acid, and a salt thereof may be mentioned.

As the diaminodisulfides or diamidocarboxydisulfides, mention may be made of those represented by the following formula:

R⁹R¹⁰N—(R¹¹)ᵤ—(CH₂)ₓ—S—S—(CH₂)_{y}—(R¹²)ᵥ—NR¹³R¹⁴

wherein

R⁹, R¹⁰, R¹³ and R¹⁴ independently denote a hydrogen atom; a C₁-C₁₈ alkyl group, preferably a C₁-C₁₀ alkyl group, and more preferably a C₁-C₆ alkyl group; a C₂-C₁₈ alkenyl group, preferably a C₂-C₁₀ alkenyl group, and more preferably a C₂-C₆ alkenyl group; a C₆-C₁₈ aryl group, preferably C₆-C₁₂ aryl group, and more preferably a C₆-C₈ aryl group; or a C₁-C₁₈ alkyl-CO— group, preferably a C₁-C₁₀ alkyl-CO— group, and more preferably a C₁-C₆ alkyl-CO— group;

R¹¹ and R¹² independently denote a C₁-C₁₈ alkylene group, preferably a C₁-C₁₀ alkylene group, and more preferably a C₁-C₆ alkylene group; a C₂-C₁₈ alkenylene group, preferably a C₂-C₁₀ alkenylene group, and more preferably a C₂-C₆ alkenylene group; a C₆-C₁₈ arylene group, preferably a C₆-C₁₂ arylene group, and more preferably a C₆-C₈ arylene group; or —CR¹⁵R¹⁶— wherein one of R¹⁵ and R¹⁶ denotes a hydrogen atom and the other denotes a carboxyl group;

u and v independently denote a number from 0 to 2, and preferably 0 or 1;

x and y independently denote a number from 0 to 5, and preferably 0 to 3, and

each of u+x and y+v is higher or equal to 1, and

salts thereof.

For example, cystine and cystamine, and a salt thereof may be mentioned.

As the disulfide, oxidized glutathione and salts thereof may also be mentioned.

It is preferable that the disulfide be selected from the group consisting of dithiodiglycolic acid, dithiodilactic acid, cystine, cystamine, disulfide of mercaptopropionic acid, and salts thereof.

The reducing composition may comprise the disulfide in an amount of from 1 to 15% by weight, preferably 1.5 to 9% by weight, and more preferably 2 to 8% by weight relative to the total weight of the composition.

The reducing composition used in the present invention also comprises at least one thiol.

The expression “thiol” here means a compound having at least one thiol group (—SH). As long as the thiol includes at least one thiol group, the type of the thiol is not limited.

The thiol may preferably be chosen from the group consisting of thioglycolic acid and derivatives thereof, in particular esters thereof such as glycerol or glycol monothioglycolate; thiolactic acid and derivatives thereof, in particular esters thereof such as glycerol monothiolactate; 3-mercaptopropionic acid and derivatives thereof, in particular esters thereof such as glycerol 3-mercaptopropionate and ethyleneglycol 3-mercaptopropionate; cysteamine and derivatives thereof, in particular C₁₋₄ acyl derivatives thereof such as N-acetylcysteamine and N-propionylcysteamine; mono-thioglycerol and derivatives thereof, in particular esters; cysteine and derivatives thereof, in particular esters such as N-acetylcysteine, N-alkanoylcysteine and cysteine alkyl esters; and salts thereof.

As the above salts, mention may be made of, for example, ammonium salts; primary-, secondary- or tertiary-amine salts; alkaline metal salts; and, alkaline earth metal salts. As the primary-, secondary- or tertiary-amine, for example, monoethanolamine, di-isopropanolamine or triethanolamine, respectively, may be mentioned.

Other suitable examples of the thiol that may be used in the reducing composition used in the present invention include, but are not limited to, sugar N-mercapto alkyl amides such as N-(mercapto-2-ethyl)gluconamide, β-mercaptopropionic acid and derivatives thereof; thiomalic acid; pantheteine; N-(mercaptoalkyl)ω-hydroxyalkyl amides such as those described in European Patent Application No. 0 354 835 and N-mono- or N,N-dialkylmercapto 4-butyramides such as those described in European Patent Application No. 0 368 763; aminomercaptoalkyl amides such as those described in European Patent Application No. 0 432 000 and alkylaminomercaptoalkylamides such as those described in European Patent Application No. 0 514 282; (2/3) hydroxy-2 propyl thioglycolate; and the hydroxy-2 methyl-1 ethyl thioglycolate-based mixture (67/33) described in French Patent Application No. 2 679 448.

It is preferable that the thiol be selected from the group consisting of thioglycolic acid, thiolactic acid, cysteine, cysteamine, mercaptopropionic acid, and salts thereof.

The reducing composition may comprise the thiol in an amount of from 1 to 20% by weight, preferably 5 to 15% by weight, and more preferably 7 to 10% by weight, relative to the total weight of the composition.

It is preferable that the disulfide/thiol combination in the reducing composition used in the present invention be chosen from the following combinations: dithiodiglycolic acid or its salts/thioglycolic acid or its salts, dithiodilactic acid or its salts/thiolactic acid or its salts, cystine/cysteine, cystamine/cysteamine, mercaptopropionic acid disulphide/mercaptopropionic acid.

According to the present invention, the weight ratio of the disulfide/thiol in the reducing composition used in the present invention is from 0.35 to 0.75, inclusive, preferably 0.4 to 0.7, and more preferably 0.45 to 0.65. If the weight ratio of the disulfide/thiol is less than 0.35, curl lastingness of the keratin fibers may become poor, even worse than traditional deformation processes; the keratin fibers may wear bad odor; and the color of the keratin fibers may fade; in particular the fixing step of the keratin fibers is performed in a short period of time or an oxidizing composition with low oxidizing power is used in the fixing step, although the curl efficiency just after the deformation process of the keratin fibers may be high (good curling). On the other hand, if the weight ratio of the disulfide/thiol is more than 0.75, the curl efficiency just after the deformation process of the keratin fibers may be low (limited curling); curl lastingness of the keratin fibers may become poor, even worse than traditional deformation processes; the keratin fibers may wear bad odor; and the color of the keratin fibers may fade; in particular the fixing step of the keratin fibers is performed in a short period of time or an oxidizing composition with low oxidizing power is used in the fixing step. In addition, if the weight ratio of the disulfide/thiol is more than 0.75, the reducing composition may be unstable such that the disulfide/thiol equilibrium shifts to increase the concentration of thiol and decrease the concentration of disulfide over time.

The composition may comprise at least one alkaline agent and/or at least one acidic agent.

The alkaline agent may be an inorganic alkaline agent. It is preferable that the inorganic alkaline agent be selected from the group consisting of ammonia; alkaline metal hydroxides; alkaline earth metal hydroxides; alkaline metal phosphates and alkaline metal monohydrogenophosphates such as sodium phosphate or sodium monohydrogeno phosphate, alkaline metal carbonates and alkaline metal monohydrogenocarbonates such as sodium carbonate or sodium monohydrogeno carbonate.

As examples of the inorganic alkaline metal hydroxides, mention may be made of sodium hydroxide and potassium hydroxide. As examples of the alkaline earth metal hydroxides, mention may be made of calcium hydroxide and magnesium hydroxide. As inorganic alkaline agent, sodium hydroxide is preferable.

The alkaline agent may be an organic alkaline agent. It is preferable that the organic alkaline agent be selected from the group consisting of monoamines and derivatives thereof; diamines and derivatives thereof; polyamines and derivatives thereof; basic amino acids and derivatives thereof; oligomers of basic amino acids and derivatives thereof; polymers of basic amino acids and derivatives thereof; urea and derivatives thereof; and guanidine and derivatives thereof.

As examples of the organic alkaline agents, mention may be made of alkanolamines such as mono-, di- and tri-ethanolamine, and isopropanolamine; urea, guanidine and their derivatives; basic amino acids such as lysine, ornithine or arginine; and diamines such as those described in the structure below:

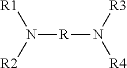

wherein R denotes an alkylene such as propylene optionally substituted by a hydroxyl or a C₁-C₄ alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof. Arginine, urea and monoethanolamine are preferable.

The alkaline agents may be used in a total amount of from 0.1 to 30% by weight, preferably from 0.2 to 20% by weight, more preferably from 0.3 to 10% by weight, relative to the total weight of the composition, depending on their solubility.

As the acidic agents, mention may be made of any inorganic or organic acids which are commonly used in cosmetic products such as citric acid, lactic acid, phosphoric acid or hydrochloric acid (HCl). Citric acid and HCl are preferable.

The amount of the acidic agent(s) is not limited, but may be from 0.1 to 30% by weight, preferably from 0.2 to 20% by weight, more preferably from 0.3 to 10% by weight, relative to the total weight of the composition, depending on their solubility.

The pH of the composition may range from 7 to 10, preferably from 8 to 9.5, and more preferably from 8.5 to 9.5. If the pH of the composition is not relatively high, damage to the keratin fibers by the composition can be further reduced. In order to adjust the pH, alkaline agent(s) and/or acidic agent(s) may be used alone or in combination.

The reducing composition used in the present invention may also comprise at least one additional ingredient. The amount of the additional ingredient(s) is not limited, but may be from 0.1 to 10% by weight relative to the total weight of the reducing composition. The additional ingredient(s) may be selected from the group consisting of volatile or non volatile, linear or cyclic, amine-type or not, silicones; cationic, anionic, non ionic or amphoteric polymers; peptides and derivatives thereof; protein hydrolyzates; synthetic or natural waxes, and especially fatty alcohols; swelling agents and penetrating agents, as well as other active compounds, such as anionic, cationic, non ionic, amphoteric or zwitterionic surfactants; agents for combating hair loss; anti-dandruff agents; associative-type or not, natural or synthetic thickeners; suspending agents; sequestering agents; opacifying agents; dyes; sunscreen agents; vitamins or provitamins; mineral, vegetable or synthetic oils; as well as fragrances; preserving agents, stabilizers; and mixtures thereof.

The vehicle for the reducing composition used in the present invention is preferably an aqueous medium consisting of water and may advantageously contain one or several cosmetically acceptable organic solvents, which particularly include alcohols, such as ethyl alcohol, isopropyl alcohol, benzyl alcohol and phenylethyl alcohol, or polyols or polyol ethers, such as ethylene glycol monomethyl, monoethyl and monobutyl ethers, propylene glycol or ethers thereof, such as propylene glycol monomethylether, butylene glycol, dipropylene glycol as well as diethylene glycol alkyl ethers, such as diethylene glycol monoethylether or monobutylether and glycerol. The water may be present in a concentration of from 5 to 90% by weight relative to the total weight of the composition. The organic solvent(s) may then be present in a concentration of from 0.1 to 20% by weight, and preferably from 1 to 10% by weight relative to the total weight of the composition.

The reducing composition used in the present invention may exist in any form such as a lotion, a gel, thickened or not, a foam, or a cream. The reducing composition may be contained in any container such as a spray bottle, a pump bottle or an aerosol.

(Keratin Fiber Treatment Process)

The process for treating keratin fibers according to the present invention can be performed by

(i) applying to the keratin fibers a reducing composition as explained above;

(ii) optionally rinsing the keratin fibers;

(iii) oxidizing the keratin fibers; and

(iv) optionally rinsing and/or drying the keratin fibers.

In the step (i), the above-described reducing composition is applied to the keratin fibers. The application of the reducing composition may be performed by any means, such as a brush and a comb. It may be possible that the keratin fibers after the application of the reducing composition be left as they are for a certain amount of time typically from 1 minute to 1 hour, preferably from 1 to 15 minutes, if necessary, in order to let the reducing composition penetrate into the keratin fibers.

According to the present invention, keratin fibers such as hair may be subjected to mechanical tension which is typically used for permanent deformation of the keratin fibers before and/or after the step (i), preferably before the step (i).

The mechanical tension can be applied to the keratin fibers by any means to deform the keratin fibers to an intended shape. For example, the mechanical tension may be provided by at least one reshaping means selected from the group consisting of a curler, a roller, a clip, a plate and an iron. The reshaping means may comprise at least one heater. If the keratin fibers are rolled around a curler, this rolling-up may be performed on the entire length of the keratin fibers or, for example, on half the length of the keratin fibers. Depending on, for example, the desired hairstyle shape and amount of curls, the rolling-up may be performed with more or less thick locks.

In the step (ii), the keratin fibers may be rinsed preferably with water.

If necessary, the keratin fibers are provided with heat, for deforming or reshaping the keratin fibers, preferably after the step (i) or the step (ii) if present. For example, the keratin fibers which may or may not be under mechanical tension are heated with a heating means such as a heating iron (when straightening) or a heating rod (when waving) at typically from 50 to 150° C., preferably 70 to 100° C. for typically 5 minutes to 2 hours, preferably 10 minutes to 1 hour.

In step (iii), the keratin fibers are oxidized. Typically, an oxidizing composition is used for this oxidizing step. The oxidizing composition comprises at least one oxidizing agent. Preferably, the oxidizing agent(s) is (are) chosen from hydrogen peroxide, urea peroxide, alkali metal bromates or ferricyanides, and peroxygenated salts, for instance alkali metal or alkaline-earth metal persulfates, perborates and percarbonates, and peracids and precursors thereof. It is preferable that the oxidizing composition comprise hydrogen peroxide, especially as an aqueous solution thereof (aqueous hydrogen peroxide solution). The concentration of the hydrogen peroxide in the oxidizing composition may range from 0.1 to 20% by weight, preferably 1 to 10% by weight, relative to the total weight of the oxidizing composition.

The oxidizing composition may be aqueous or nonaqueous. The term “aqueous” means that the developer comprises more than 5% by weight of water, preferably more than 10% by weight of water and even more advantageously more than 20% by weight of water.

Usually, the pH of the oxidizing composition, when it is aqueous, is less than 7.

The oxidizing composition may also contain at least one additional ingredient explained in detail previously in the context of the reducing composition.

The oxidizing composition may be in various forms, for instance a shampoo, a gel, a mousse, a thickened lotion and a cream.

According to the present invention, it is preferable that the step (iii) performs mild oxidation by applying an oxidizing composition with a short, or with no, standing time on the keratin fibers. As the standing time, mention may be made of 3 minutes to 30 minutes, preferably 5 minutes to 15 minutes.

In step (iv), the keratin fibers may be rinsed preferably with water, and/or may be dried. The drying of the keratin fibers can be performed with a conventional drying means such as a hair drier.

It is possible for the reducing and oxidizing compositions used in the present invention be in the form of a kit. The kit may be for deforming keratin fibers, comprising:

the reducing composition as explained above; and

the oxidizing composition as explained above,

wherein

the reducing and oxidizing compositions are separated into two or more compartments in the kit.

### EXAMPLES

The present invention will be described in more detail by way of examples, which however should not be construed as limiting the scope of the present invention.

### Example 1

The following compositions were prepared (the unit of the content is grams based on material as provided).

Composition 1 (Reducing Lotion)

TABLE 1

| | wt % |
|---|---|
| Ammonium thioglycolate aqueous solution | 16 |
| (at 60% of TGA eq.) | |
| Ammonium dithiodiglycolate aqueous | 12 |
| solution | |
| (at 40% of disulfide eq.) | |
| Pentasodium pentetate (40%) aqueous solution | 0.4 |
| Monoethanolamine | 3.7 |
| Ammonia (20%) aqueous solution | 3.3 |
| Water | qsp 100 |
| pH | 9 |
| Ratio disulfide/thiol | 0.5 |

Composition 2 (Oxidizing Lotion)

TABLE 2

| | wt % |
|---|---|
| Hydrogen peroxide (35% in water) | 6.85 |
| Tetra sodium pyrophosphate | 0.04 |
| Sodium salicylate | 0.04 |
| Tetra sodium editronate (30% in water) | 0.2 |
| Lauramine oxide (30% in water) | 3.6 |
| Water | qsp 100 |

Composition 1 was applied onto a 1 g Japanese hair swatch which had been previously wrapped on a 1.7 cm perm-roller in a weight ratio of 1:2 of the swatch to Composition 1, and left for 15 minutes. After a pausing time, the swatch was rinsed. Then, Composition 2 was applied onto the swatch in a weight ratio of 1:2 of the swatch to Composition 2, and left for 10 minutes. Then, the perm-roller was removed and the swatch was rubbed to spread homogeneously Composition 2 onto the hair. Then, the swatch was rinsed and dried.

The degree of curling was very good right after the treatments by Compositions 1 and 2. The good curling was maintained even after 5 shampoos. No particular strong odor was noticed on the hair after the treatments.

### Comparative Example 1

The following compositions were prepared (the unit of the content is grams based on material as provided).

Composition 3 (Reducing Lotion)

TABLE 3

| | wt % |
|---|---|
| Ammonium thioglycolate aqueous solution | 15 |
| (at 60% of TGA eq.) | |
| Ammonium dithiodiglycolate aqueous solution | 18 |
| (at 40% of disulfide eq.) | |
| Pentasodium pentetate (40%) aqueous solution | 0.4 |
| Monoethanolamine | 3.7 |
| Ammonia (20%) aqueous solution | 3.3 |
| Water | qsp 100 |
| pH | 9 |
| Ratio disulfide/thiol | 0.8 |

Composition 3 was applied onto a 1 g Japanese hair swatch which had been previously wrapped on a 1.7 cm perm-roller in a weight ratio of 1:2 of the swatch to Composition 3, and left for 15 minutes. After a pausing time, the swatch was rinsed. Then, Composition 2 was applied onto the swatch in a weight ratio of 1:2 of the swatch to Composition 2, and left for 10 minutes. Then, the perm-roller was removed and the swatch was rubbed to spread homogeneously Composition 2 onto the hair. Then, the swatch was rinsed and dried.

The curl efficiency was weaker than in the Example 1.

### Comparative Example 2

The following compositions were prepared (the unit of the content is grams based on material as provided).

Composition 4 (Reducing Lotion)

TABLE 4

| | wt % |
|---|---|
| Ammonium thioglycolate aqueous solution | 15 |
| (at 60% of TGA eq.) | |
| Ammonium dithiodiglycolate aqueous solution | 6.75 |
| (at 40% of disulfide eq.) | |
| Pentasodium pentetate (40%) aqueous solution | 0.4 |
| Monoethanolamine | 3.7 |
| Ammonia (20%) aqueous solution | 3.3 |
| Water | qsp 100 |
| pH | 9 |
| Ratio disulfide/thiol | 0.3 |

Composition 4 was applied onto a 1 g Japanese hair swatch which had been previously wrapped on a 1.7 cm perm-roller in a weight ratio of 1:2 of the swatch to Composition 4, and left for 15 minutes. After a pausing time, the swatch was rinsed. Then, Composition 2 was applied onto the swatch in a weight ratio of 1:2 of the swatch to Composition 2, and left for 10 minutes. Then, the perm-roller was removed and the swatch was rubbed to spread homogeneously Composition 2 onto the hair. Then, the swatch was rinsed and dried.

The curl efficiency was strong just after the treatments, but strong odor was noticed on the hair. The degree of curl was rapidly lost with shampoos.

1-16. (canceled)

17. A process for treating keratin fibers, the method comprising:
(i) applying to the keratin fibers a reducing composition comprising a disulfide/thiol combination comprising at least one disulfide and at least one thiol, wherein the weight ratio of disulfide to thiol in the disulfide/thiol combination ranges from about 0.35 to about 0.75;
(ii) optionally rinsing the keratin fibers;
(iii) oxidizing the keratin fibers; and
(iv) optionally rinsing and/or drying the keratin fibers.

18. The process according to claim 17, wherein the weight ratio of disulfide to thiol ranges from about 0.4 to about 0.7.

19. The process according to claim 17, wherein the weight ratio of disulfide to thiol ranges from about 0.45 to about 0.65.

20. The process according to claim 17, wherein the disulfide is chosen from: dithiodiglycolic acid, dithiodilactic acid, cystine, cystamine, disulfide of mercaptopropionic acid, salts thereof, and combinations thereof.

21. The process according to claim 17, wherein the disulfide is present in the reducing composition in an amount ranging from about 1 to about 15% by weight, relative to the total weight of the composition.

22. The process according to claim 17, wherein the disulfide is present in the reducing composition in an amount ranging from about 1.5 to about 9% by weight, relative to the total weight of the composition.

23. The process according to claim 17, wherein the thiol is chosen from: thioglycolic acid, thiolactic acid, cysteine, cysteamine, mercaptopropionic acid, salts thereof, and combinations thereof.

24. The process according to claim 17, wherein the thiol is present in the reducing composition in an amount ranging from about 1 to about 20% by weight, relative to the total weight of the composition.

25. The process according to claim 17, wherein the thiol is present in the reducing composition in an amount ranging from about 5 to about 15% by weight, relative to the total weight of the composition.

26. The process according to claim 17, wherein the disulfide/thiol combination is chosen from: dithiodiglycolic acid or its salts/thioglycolic acid or its salts; dithiodilactic acid or its salts/thiolactic acid or its salts; cystine/cysteine; cystamine/cysteamine; and mercaptopropionic acid disulphide/mercaptopropionic acid.

27. The process according to claim 17, wherein the pH of the reducing composition ranges from about 7 to about 10.

28. The process according to claim 17, wherein the pH of the reducing composition ranges from about 8 to about 9.5.

29. The process according to claim 17, wherein the reducing composition further comprises at least one alkali agent.

30. The process according to claim 29, wherein the at least one alkali agent is an inorganic alkaline agent.

31. The process according to claim 30, wherein the inorganic alkaline agent is chosen from: ammonia, alkaline metal hydroxides, alkaline earth metal hydroxides, alkaline metal phosphates and alkaline metal monohydrogenophosphates, alkaline metal carbonates and alkaline metal monohydrogenocarbonates, and combinations thereof.

32. The process according to claim 29, wherein the at least one alkali agent is an organic alkaline agent.

33. The process according to claim 32, wherein the organic alkaline agent is chosen from: monoamines and derivatives thereof, diamines and derivatives thereof, polyamines and derivatives thereof, basic amino acids and derivatives thereof, oligomers of basic amino acids and derivatives thereof, polymers of basic amino acids and derivatives thereof, urea and derivatives thereof, guanidine and derivatives thereof, and combinations thereof.

34. The process according to claim 17, wherein oxidizing the keratin fibers comprises applying an oxidizing composition with a short standing time, or with no standing time, on the keratin fibers.

35. The process according to claim 34, wherein the oxidizing composition comprises an aqueous hydrogen peroxide solution.

36. The process according to claim 35, wherein the hydrogen peroxide is present in the oxidizing composition in an amount ranging from about 0.1 to about 20% by weight, relative to the total weight of the oxidizing composition.

37. The process according to claim 35, wherein the hydrogen peroxide is present in the oxidizing composition in an amount ranging from about 1 to about 10% by weight, relative to the total weight of the oxidizing composition.

38. The process according to claim 34, wherein the oxidizing composition is in the form of a shampoo, a gel, a mousse, a thickened lotion, or a cream.
